(19) United States
(12) Patent Application Publication
(10) Pub. No.: US 2022/0192519 A1
(43) Pub. Date: Jun. 23, 2022

(54) PHYSIOLOGICAL INFORMATION PROCESSING DEVICE, PHYSIOLOGICAL INFORMATION PROCESSING METHOD, PROGRAM AND STORAGE MEDIUM

(71) Applicant: NIHON KOHDEN CORPORATION, Tokyo (JP)

(72) Inventors: Yoshiharu HARADA, Tokorozawa-shi (JP); Yoshihiro SUGO, Tokorozawa-shi (JP); Sayaka YAMAZAKI, Tokorozawa-shi (JP); Makiko FUMOTO, Tokorozawa-shi (JP)

(73) Assignee: NIHON KOHDEN CORPORATION, Tokyo (JP)

(21) Appl. No.: 17/553,636

(22) Filed: Dec. 16, 2021

(30) Foreign Application Priority Data
Dec. 18, 2020 (JP) 2020-210400

(51) Int. Cl. A61B 5/029; A61B 5/02; A61B 5/00

(52) U.S. Cl. CPC A61B 5/029; A61B 5/7275; A61B 5/743; A61B 5/02007

(57) ABSTRACT

A physiological information processing method includes a step of acquiring a parameter related to an amount of blood delivered from a heart of a subject or stiffness of a blood vessel of the subject; a step of displaying a trend graph indicating a change over time in the parameter in a graph display region; a step of determining a reference value of the parameter in accordance with an input operation of a user; and a step of displaying a reference guideline indicating the reference value of the parameter in the graph display region.

The present application claims the benefit of priority of Japanese Patent Application No. 2020-210400, filed on Dec. 18, 2020, the content of which is incorporated herein by reference.

### TECHNICAL FIELD

The present disclosure relates to a physiological information processing device and a physiological information processing method. Further, the present disclosure relates to a program configured to cause a computer to execute the physiological information processing method and a computer-readable storage medium in which the program is stored.

### BACKGROUND

JP-T-2014-518715 discloses a vital monitor that displays a trend graph indicating a change over time in a cardiac output of a patient. A health professional can grasp a state of a cardiac function of a subject by observing the trend graph indicating the change over time in the cardiac output of the patient.

### SUMMARY

Incidentally, there may be a case where a health professional administers a fluid to a patient in order to recover a cardiac function of the patient. In this case, in order to check fluid responsiveness of the patient, it is necessary for the health professional to check whether a cardiac output of the patient appropriately increases after the fluid is administered to the patient. However, the health professional may not be capable of clearly grasping whether the cardiac output of the patient increases after the fluid is administered to the patient only by checking a trend graph indicating a change over time in the cardiac output. From the above viewpoint, there is room for improving usability of a vital monitor.

The present disclosure is provided with a physiological information processing method and a physiological information processing device capable of more accurately grasping a state of a cardiac function of a subject.

A first aspect of the present disclosure of a physiological information processing method includes:

acquiring a parameter related to an amount of blood delivered from a heart of a subject or stiffness of a blood vessel of the subject;

displaying a trend graph indicating a change over time in the parameter in a graph display region;

determining a reference value of the parameter in accordance with an input operation of a user; and

displaying a reference guideline indicating the reference value of the parameter in the graph display region.

A program configured to cause a computer to execute the physiological information processing method and a computer-readable storage medium in which the program is stored are also provided.

A second aspect of the present disclosure of a physiological information processing device includes:

one or more processors; and

one or more memories configured to store a computer readable command, wherein

when the computer readable command is executed by the one or more processors, the physiological information processing device

acquires a parameter related to an amount of blood delivered from a heart of a subject or stiffness of a blood vessel of the subject,

displays a trend graph indicating a change over time in the parameter in a graph display region,

determines a reference value of the parameter in accordance with an input operation of a user, and

displays a reference guideline indicating the reference value of the parameter in the graph display region.

According to the present disclosure, it is possible to provide a physiological information processing method and a physiological information processing device capable of more accurately grasping a state of a cardiac function of a subject.

### BRIEF DESCRIPTION OF DRAWINGS

FIG. 1 illustrates an example of a hardware configuration of a physiological information processing device according to the presently disclosed subject matter.

FIG. 2 is a flowchart illustrating a process of updating a trend graph of a cardiac output CO and a trend graph of a stroke volume variation SVV based on physiological information data acquired from a physiological information sensor.

FIG. 3 illustrates an example of the trend graph of the cardiac output CO and the trend graph of the stroke volume variation SVV displayed in a graph display region.

FIG. 4 is a flowchart illustrating a process of displaying a reference guideline and a reference guide region in the graph display region.

FIG. 5 illustrates an example of the trend graph of the cardiac output CO, the trend graph of the stroke volume variation SVV, the reference guideline, and the reference guide region displayed in the graph display region.

FIG. 6 illustrates an example of an input operation screen displayed on a display unit.

### DESCRIPTION OF EMBODIMENTS

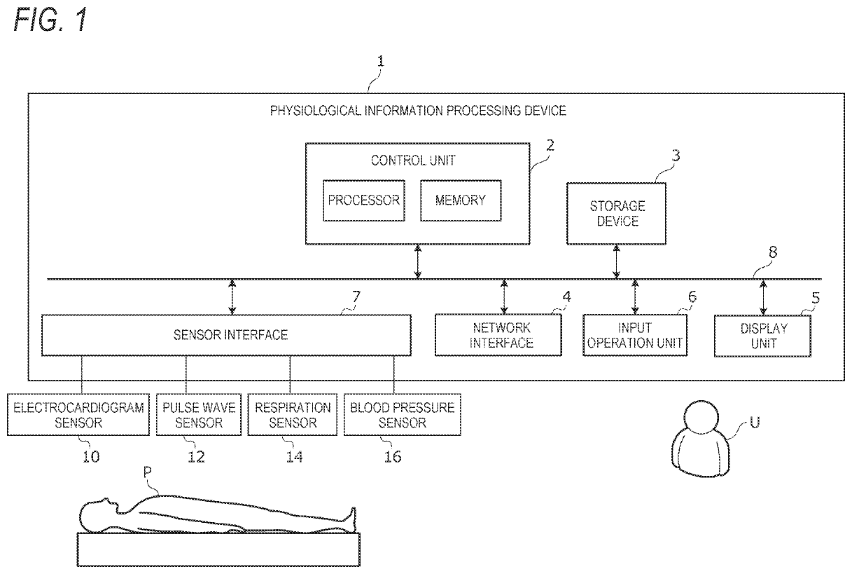

Hereinafter, the present embodiment will be described with reference to the drawings. First, a hardware configuration of a physiological information processing device 1 according to the presently disclosed subject matter (hereinafter, simply referred to as the present embodiment) will be described with reference to FIG. 1.

FIG. 1 illustrates an example of the hardware configuration of the physiological information processing device 1 according to the present embodiment. As illustrated in FIG. 1, the physiological information processing device 1 (hereinafter, simply referred to as “processing device 1”) can include a control unit 2, a storage device 3, a network interface 4, a display unit 5, an input operation unit 6, and a sensor interface 7. Such components are connected to each other via a bus 8 so as to be capable of communicating with each other.

The processing device 1 may be a patient monitor (for example, a bedside monitor, a central monitor, a vital sign telemeter, a medical telemeter, or the like) configured to display a trend graph of vital signs of a subject P. The processing device 1 may also be a personal computer, a workstation, a smartphone, a tablet, or a wearable device (for example, a smart watch, an AR glass, or the like) attached to a body (for example, an arm, a head, or the like) of a health professional U.

The control unit 2 can include at least one memory and at least one processor. Each memory is configured to store a computer readable command (program). For example, the memory may be constituted by a read only memory (ROM) where various programs and the like are stored, a random access memory (RAM) including a plurality of work areas where various programs and the like executed by the processor are stored, and the like. The memory, may also be constituted by a flash memory or the like, Each processor is constituted by, for example, at least one of a central processing unit (CPU), a micro processing unit (MPU), and a graphics processing unit (GPU). The CPU may be configured with a plurality of CPU cores. The GPU may be configured with a plurality of GPU cores. The processor may be configured to load a program specified from various programs incorporated in the storage device 3 or the ROM onto the RAM and execute various processes in cooperation with the RAM.

The processor may load a physiological information processing program to be described later onto the RAM and execute the program in cooperation with the RAM such that the control unit 2 may control various operations of the processing device 1. Details of the physiological information processing program will be described later.

The storage device 3 is, for example, a storage device (storage) such as a hard disk drive (HDD), a solid state drive (SSD), or a flash memory, and is configured to store programs and various types of data. The physiological information processing program may be incorporated in the storage device 3. In addition, physiological information data such as electrocardiogram data, blood pressure data, pulse wave data, and respiratory waveform data of the subject P may be stored in the storage device 3. For example, electrocardiogram data acquired by an electrocardiogram sensor 10 may be stored in the storage device 3 via the sensor interface 7.

The network interface 4 is configured to connect the processing device 1 to a communication network. Specifically, the network interface 4 may include various wired connection terminals in order to communicate with an external device such as a server via the communication network. In addition, the network interface 4 may include various processing circuits, antennas, and the like in order to perform wireless communication with an access point. A wireless communication standard between the access point and the processing device 1 is, for example, Wi-Fi (registered trademark), Bluetooth (registered trademark), ZigBee (registered trademark), LPWA, or a fifth generation mobile communication system (5G). The communication network is a local area network (LAN), a wide area network (WAN), the Internet, or the like. For example, the physiological information processing program and the physiological information data may be acquired from a server arranged on the communication network via the network interface 4.

The display unit 5 may be a display device such as a liquid crystal display or an organic EL display, or may be a display device such as a transmissive or non-transmissive head-mounted display mounted on a head of an operator. Further, the display unit 5 may also be a projector device that projects an image onto a screen.

The input operation unit 6 is configured to receive an input operation of the health professional U (an example of a user) who operates the processing device 1, and to generate an instruction signal corresponding to the input operation. The input operation unit 6 is, for example, a touch panel overlaid on the display unit 5, an operation button attached to a housing, a mouse, and/or a keyboard. After the instruction signal generated by the input operation unit 6 is transmitted to the control unit 2 via the bus 8, the control unit 2 performs a predetermined operation in accordance with the instruction signal.

The sensor interface 7 is an interface configured to communicably connect physiological information sensors such as the electrocardiogram sensor 10, a pulse wave sensor 12, a respiration sensor 14, and a blood pressure sensor 16 to the processing device 1. The sensor interface 7 may include an input terminal to which physiological information data output from these physiological information sensors is input. Specifically; the input terminal may be a multi-connector that can be physically connected to a plurality of types of physiological information sensors each of which has a different number of pins. The input terminal may be physically connected to a connector of each physiological information sensor. The sensor interface 7 may also include a wireless communication circuit, an antenna, and the like so as to perform wireless communication with these physiological information sensors. The sensor interface 7 may also include an analog processing circuit (for example, a filter circuit, an amplifier circuit, an AD converter, or the like) configured to process a signal output from each physiological information sensor. In this way, an analog signal output from the physiological information sensor may be converted into a digital signal by the sensor interface 7.

The electrocardiogram sensor 10 is configured to acquire electrocardiogram data indicating an electrocardiogram waveform of the subject P. The pulse wave sensor 12 (for example, a SpO2 sensor) is configured to acquire pulse wave data indicating a pulse wave of the subject P. The respiration sensor 14 is configured to acquire respiratory waveform data indicating a respiratory waveform (for example, a capnogram) of the subject P. The blood pressure sensor 16 is configured to invasively or non-invasively acquire blood pressure data indicating a change over time in blood pressure (in particular, arterial pressure and/or venous pressure) of the subject P. In this regard, the blood pressure sensor 16 may be constituted by a plurality of blood pressure sensors. Specifically; the blood pressure sensor 16 may include a blood pressure sensor configured to acquire blood pressure data indicating a change over time in arterial pressure, and a blood pressure sensor configured to acquire blood pressure data indicating a change over time in venous pressure.

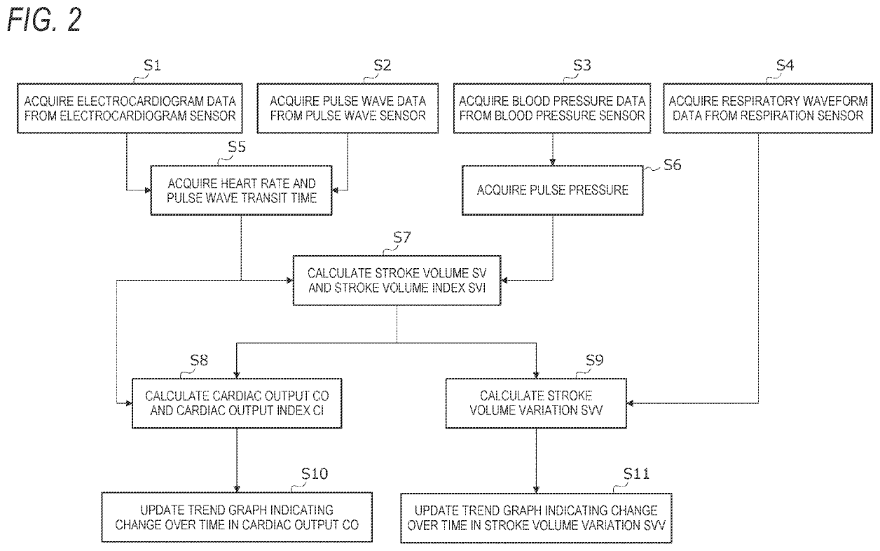

Next, with reference to FIG. 2, a process of updating a trend graph of a cardiac output CO and a trend graph of a stroke volume variation SVV based on the physiological information data acquired from the physiological information sensors (the electrocardiogram sensor 10, the pulse wave sensor 12, the respiration sensor 14, and the blood pressure sensor 16) will be described below. FIG. 2 is a flowchart illustrating the process of updating the trend graph of the cardiac output CO and the trend graph of the stroke volume variation SVV based on the physiological information data acquired from each physiological information sensor.

As illustrated in FIG. 2, in step S1, the control unit 2 acquires the electrocardiogram data of the subject P from the electrocardiogram sensor 10 via the sensor interface 7. Next, in step S2, the control unit 2 acquires the pulse wave data from the pulse wave sensor 12 via the sensor interface 7. In step S3, the control unit 2 acquires the blood pressure data from the blood pressure sensor 16 via the sensor interface 7. In step S4, the control unit 2 acquires the respiratory waveform data from the respiration sensor 14 via the sensor interface 7.

Next, in step S5, the control unit 2 acquires a heart rate HR of the subject P based on the acquired electrocardiogram data. Specifically, the control unit 2 specifies an RR interval indicating a time interval between adjacent R waves from the electrocardiogram data. Thereafter, the control unit 2 calculates a moving average value of the RR interval, and acquires the heart rate HR (bpm) of the subject P from the moving average value of the RR interval based on a relational expression of “heart rate=60/RR interval”. The control unit 2 may acquire the moving average value of the heart rate FIR every predetermined period (for example, every one second).

Further, the control unit 2 acquires a pulse wave transit time (hereinafter, referred to as PWTT) based on the electrocardiogram data and the pulse wave data. Here, the PWTT refers to a time interval from a peak point of a predetermined R wave of an electrocardiogram to a rising point of a predetermined pulse wave waveform appearing next to the predetermined R wave. In this regard, the control unit 2 specifies a time corresponding to the peak point of the predetermined R wave from the electrocardiogram data, and specifies a time corresponding to the rising point of the predetermined pulse wave waveform appearing next to the predetermined R wave from the pulse wave data. Next, the control unit 2 measures the PIA/TT by calculating a time interval between the time of the rising point of the predetermined pulse wave waveform and the time of the peak point of the predetermined R wave. Thereafter, the control unit 2 calculates a moving average value of the PWTT. The control unit 2 may acquire the moving average value of the PWTT every predetermined period (for example, every one second).

Next, in step S6, the control unit 2 acquires pulse pressure PP of the subject P from the blood pressure data. In particular, the control unit 2 specifies systolic blood pressure and diastolic blood pressure based on the blood pressure data, and then acquires the pulse pressure PP based on the systolic blood pressure and the diastolic blood pressure. Thereafter, the control unit 2 calculates a moving average value of the pulse pressure PR The control unit 2 may acquire the moving average value of the pulse pressure PP every predetermined period (tier example, every one second).

Next, in step S7, the control unit 2 calculates a stroke volume SV (mL) and a stroke volume index SVI (mL/m²) of the subject P. Here, the calculated stroke volume SV and stroke volume index SVI may be referred to as esSV and esSVI, respectively. Specifically, the control unit 2 calculates the stroke volume SV based on the P \NTT and the following relational expression (1). Here, a is a fixed value (for example, −0.3). β and K are values of coefficients determined by calibration.

SV=K₀×(α×PWTT+β) (1)

The coefficient β may be determined by measured values of the pulse pressure PP and the PWTT. The coefficient K may be determined by measured values of the pulse pressure PP and the cardiac output CO. For a specific example of a method of calculating the fixed value a and the coefficients K and β, refer to, for example, Japanese Patent No. 4742644 which is incorporated by reference. In this way, since the values of α, β, and K are determined, it is possible to calculate the stroke volume SV from the relational expression (1) and the PWTT.

The control unit 2 also calculates the stroke volume index SVI based on the calculated SV and the following relational expression (2). Here, BSA (m²) corresponds to a body surface area of the subject P determined based on a height and a weight of the subject P. The health professional U inputs attribute data such as the height and the weight of the subject P to the processing device 1 in advance through the input operation unit 6.

SVI=SV/BSA (2)

Next, in step S8, the control unit 2 calculates the cardiac output CO (L/min) and a cardiac output index CI (L/min/m²) of the subject P. Here, the calculated cardiac output CO and cardiac output index CI may be referred to as esCCO and esCCI, respectively. Specifically, the control unit 2 calculates the cardiac output CO based on the SV calculated by the relational expression (1), the heart rate HR, and the following relational expression (3). The control unit 2 also calculates the cardiac output index CI based on the CO calculated by the relational expression (3) and the following relational expression (4).

CO=HR×SV (3)

CI=CO/BSA (4)

The control unit 2 may also calculate the stroke volume SV, the stroke volume index SVI, the cardiac output CO, and the cardiac output index CI every predetermined period (for example, every one second). The stroke volume SV, the stroke volume index SVI, the cardiac output CO, and the cardiac output index CI are parameters related to blood delivered from a heart of the subject P.

Next, in step S9, the control unit 2 calculates a stroke volume variation SVV (%) of the subject P. Specifically, the control unit 2 calculates the stroke volume variation SVV based on the stroke volume SV calculated by the relational expression (1), the respiratory waveform data, and the following relational expression (5). Here, the calculated stroke volume variation SVV may be referred to as esSVV. The control unit 2 may also calculate a moving average value of the SVV.

SVV=(SVₘₐₓ−SVₘᵢₙ)/SVₐᵥₑ×100% (5)

Here, SVₘₐₓ refers to a maximum value of the stroke volume SV during one respiration cycle. SVₘᵢₙ refers to a minimum value of the stroke volume SV during one respiration cycle. SVₐᵥₑ refers to an average value of the stroke volume SV during one respiration cycle.

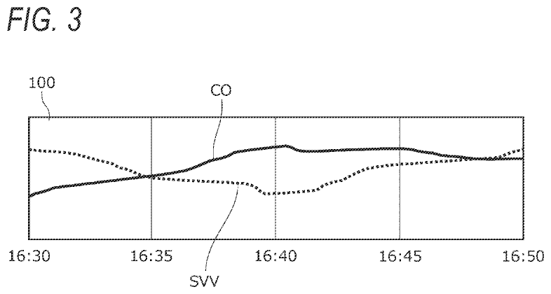

Next, in step S10, the control unit 2 updates a trend graph (an example of a second trend graph) indicating a change over time in the cardiac output CO displayed in a graph display region 100 based on the newly calculated cardiac output CO (see FIG. 3). As illustrated in FIG. 3, a horizontal axis of the graph display region 100 indicates time, while vertical axes (a left vertical axis and a right vertical axis) of the graph display region 100 indicate values of the cardiac output CO and the stroke volume variation SVV. The graph display region 100 is displayed on the display unit 5. Specifically; the graph display region 100 is a partial region of a physiological information display screen displayed on the display unit 5, and is a region for displaying trend graphs. It should be noted that, in the present embodiment, illustration of the physiological information display screen other than the graph display region 100 is omitted in order to simplify the description.

Next, in step S11, the control unit 2 updates a trend graph (an example of a first trend graph) indicating a change over time in the stroke volume variation SVV displayed in the graph display region 100 based on the newly calculated stroke volume variation SVV (see FIG. 3). The control unit 2 simultaneously displays the trend graph of the cardiac output CO and the trend graph of the stroke volume variation SVV in the graph display region 100. In this way, the health professional U can more accurately grasp a state of a cardiac function of the subject P by checking both the trend graph of the cardiac output CO and the trend graph of the stroke volume variation SVV simultaneously displayed in the graph display region 100.

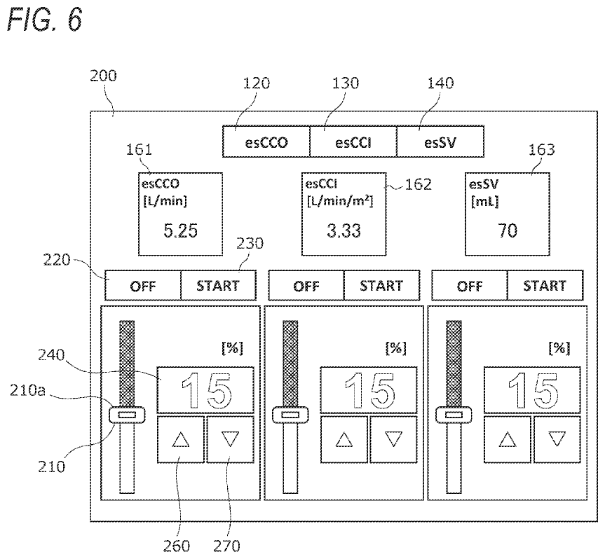

Although the trend graph of the cardiac output CO and the trend graph of the stroke volume variation SVV are displayed in the graph display region 100 in the present example, the control unit 2 may switch the trend graph displayed in the graph display region 100 in accordance with an input operation of the health professional U (user). In this regard, through an input operation of the health professional U performed on a button 120 of an input operation screen 200 illustrated in FIG. 6, the control unit 2 displays the trend graph of the cardiac output CO and the trend graph of the stroke volume variation SVV in the graph display region 100. In addition, through an input operation of the health professional U performed on a button 130 of the input operation screen 200, the control unit 2 displays the trend graph of the cardiac output index CI and the trend graph of the stroke volume variation SW in the graph display region 100. In addition, through an input operation of the health professional U performed on a button 140 of the input operation screen 200, the control unit 2 displays the trend graph of the stroke volume SV and the trend graph of the stroke volume variation SVV in the graph display region 100.

In this regard, in a case where the heart rate I-IR of the subject P is not normal, the health professional U may be capable of accurately grasping the state of the cardiac function of the subject P by checking the trend graph of the stroke volume SV rather than the trend graph of the cardiac output CO. In addition, in a case where the height or the weight of the subject P is greatly different from a standard height or a standard weight, the health professional U may be capable of more accurately grasping the state of the cardiac function of the subject P by checking the trend graph of the cardiac output index CI rather than the trend graph of the cardiac output CO.

(Reference Guideline and Reference Guide Region)

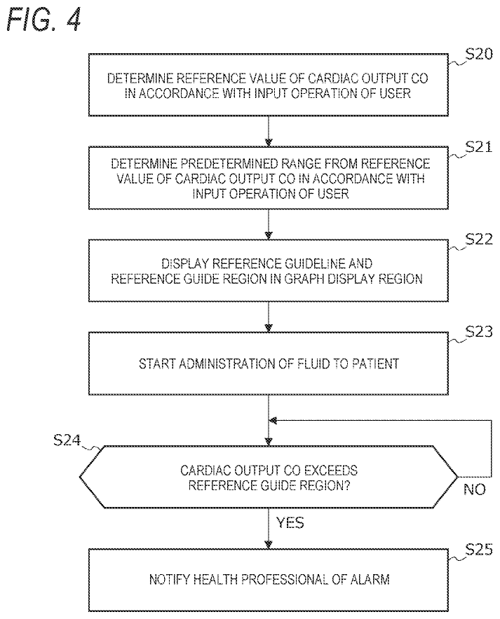

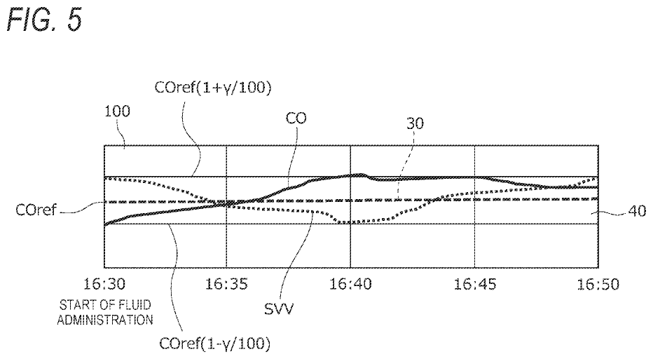

Next, a process of displaying a reference guideline 30 and a reference guide region 40 in the graph display region 100 will be described below with reference to FIGS. 4 and 5. FIG. 4 is a flowchart illustrating the process of displaying the reference guideline 30 and the reference guide region 40 in the graph display region 100, FIG. 5 illustrates an example of the trend graph of the cardiac output CO, the trend graph of the stroke volume variation SVV, the reference guideline 30, and the reference guide region 40 displayed in the graph display region 100. Although the trend graph of the cardiac output CO is displayed in the graph display region 100 in the present example, the trend graph of the stroke volume SV or the trend graph of the cardiac output index CI may be displayed in the graph display region 100 together with the trend graph of the stroke volume variation SVV instead of the trend graph of the cardiac output CO.

As illustrated in FIG. 4, in step S20, the control unit 2 determines a reference value of the cardiac output CO in accordance with an input operation of the health professional U. Specifically, the reference value CO_{ref} of the cardiac output CO is determined through an operation of the health professional U performed on a start button 230 displayed on the input operation screen 200 illustrated in FIG. 6. A current value of the cardiac output CO when the start button 230 is operated by the health professional U is set as the reference value CO_{ref} of the cardiac output CO. For example, when the cardiac output CO is 5.25 (L/min) when the start button 230 is operated by the health professional U, the reference value CO_{ref} of the cardiac output CO is 5.25 (L/min). As will be described later, the reference value CO_{ref} is a calculated value of the cardiac output CO calculated when the start button 230 is operated before start of fluid administration.

The calculated current value of the cardiac output CO is displayed in a display region 161 of the input operation screen 200. A current value of the calculated cardiac output index CI is displayed in a display region 162. A current value of the calculated stroke volume SV is displayed in a display region 163.

Next, in step S21, the control unit 2 determines a predetermined range γ from the reference value CO_{ref} of the cardiac output CO in accordance with an input operation of the health professional U. Specifically, the predetermined range γ from the reference value CO_{ref} is determined through an input operation of the health professional U performed on an adjustment slider 210 displayed on the input operation screen 200. For example, when a knob portion 210a is located at a center of the adjustment slider 210 during initial setting, the predetermined range γ may be set to 15%. A value of the predetermined range γ may be displayed in a display region 240. Further, the value of the predetermined range γ is decreased by moving the knob portion 210a downward, while the value of the predetermined range γ is increased by moving the knob portion 210a upward. In this way; the health professional U can set the predetermined range γ to any value as desired by operating the knob portion 210a.

The health professional U can also increase the value of the predetermined range γ by operating an up button 260 instead of the adjustment slider 210. In addition, the health professional UI can reduce the value of the predetermined range γ by operating a down button 270. In this way, the health professional U can set the predetermined range γ to any value as desired by operating the up button 260 or the down button. 270.

Next, in step S22, the control unit 2 displays the reference guideline 30 and the reference guide region 40 in the graph display region 100. As illustrated in FIG. 5, the control unit 2 displays the reference guideline 30, the reference guide region 40, the trend graph of the cardiac output CO, and the trend graph of the stroke volume variation SVV in the graph display region 100.

The reference guideline 30 indicates the reference value CO_{ref} of the cardiac output CO, and is a straight line extending parallel to a time axis which is a horizontal axis. When a variable on a vertical axis of the graph display region 100 is CO, the reference guideline 30 is a straight line indicated by CO=CO_{ref}. The reference guideline 30 may be colored in a color different from a background color of the graph display region 100. In addition, a display color of the reference guideline 30 may be different from a display color of the trend graph of the cardiac output CO and a display color of the trend graph of the stroke volume variation SVV.

The reference guide region 40 is a region indicating the predetermined range γ from the reference value CO_{ref}. Specifically; the reference guide region 40 indicates a region between (1−γ/100) CO_{ref} and (1+γ/100) CO_{ref}. When the variable of the vertical axis of the graph display region 100 is CO, the reference guide region 40 is a region represented by (1−γ/100) CO_{ref}≤CO≤(1+γ/100) CO_{ref}. The reference guide region 40 may be colored in a color different from the background color of the graph display region 100. In addition, a display color of the reference guide region 40 may be different from the display color of the trend graph of the cardiac output CO and the display color of the trend graph of the stroke volume variation SVV.

Next, in step S23, the health professional U starts administration of a fluid to the subject P (patient). In this example, as illustrated in FIG. 6, the administration of the fluid is disclosed at a time point 16:30, In this case, the reference value CO_{ref} corresponds to the value of the cardiac output CO of the subject P when the start button 230 is operated by the health professional U before the time point 16:30.

Next, the control unit 2 determines whether the value of the cardiac output CO exceeds the reference guide region 40 (step S24). When it is determined that the value of the cardiac output CO exceeds the reference guide region 40 (YES in step S24), the control unit 2 visually and/or audibly notifies the health professional U of an alarm (step S25). For example, the control unit 2 may display alarm information in the graph display region 100, or may output an alarm sound through a speaker (not illustrated) provided in the processing device 1. In addition, when the processing device 1 is a bed side monitor, the control unit 2 may transmit a message indicating that the value of the cardiac output CO exceeds the reference guide region 40 to a central monitor. On the other hand, when the determination result of step S24 is NO, the determination process of step S24 may be repeatedly performed. Although the process of step S24 is performed after the process of step S23 in this example, the process of step S24 may also be performed before the administration of the fluid to the patient is started.

In this example, the reference guideline 30 and the reference guide region 40 are deleted from the graph display region 100 through an operation of the health professional U performed on an OFF button 220 displayed on the input operation screen 200 illustrated in FIG. 6.

In the present example, since the trend graph of the cardiac output CO is displayed in the graph display region 100, the reference guideline 30 indicates the reference value of the cardiac output CO, and the reference guide region 40 is a region indicating the predetermined range γ from the reference value of the cardiac output CO. On the other hand, when the trend graph of the cardiac output index CI is displayed in the graph display region 100 through the input operation of the health professional U performed on the button 130, the reference guideline 30 indicates a reference value of the cardiac output index CI, and the reference guide region 40 is a region indicating the predetermined range γ from the reference value of the cardiac output index Same or similarly, when the trend graph of the stroke volume SV is displayed in the graph display region 100 through the input operation of the health professional U performed on the button 140, the reference guideline 30 indicates a reference value of the stroke volume SV, and the reference guide region 40 is a region indicating the predetermined range γ from the reference value of the stroke volume SV.

According to the present embodiment, the health professional U can more accurately, grasp the state of the cardiac function of the subject P by comparing the reference guideline 30, the reference guide region 40, the trend graph of the cardiac output CO, and the trend graph of the stroke volume variation SW displayed in the graph display region 100. For example, the health professional U can clearly grasp whether the trend graph of the cardiac output CO is appropriately raised after the fluid is administered to the subject P by comparing the trend graph of the cardiac output CO with the reference guideline 30 indicating the reference value CO_{ref} that is the value of the cardiac output CO before the fluid is administered. In this way, the health professional U can more accurately grasp the state of the cardiac function of the subject P (in particular, fluid responsiveness). Further, by comparing the trend graph of the cardiac output CO with the reference guide region 40, the health professional U can grasp whether the trend graph is excessively raised or lowered beyond a predetermined range of ±γ% (for example, ±15%) from the reference value CO_{ref} after the fluid is administered to the subject P, In this way; the health professional U can adjust an amount of the fluid administered to the subject P to an appropriate amount, and therefore, it is possible to suitably prevent the cardiac function of the subject P from being adversely affected by excessive administration of the fluid.

In order to achieve the processing device 1 according to the present embodiment by software, the physiological information processing program may be incorporated in the storage device 3 or the ROM in advance. Alternatively, the physiological information processing program may be stored in a computer-readable storage medium such as a magnetic disk (for example, an HDD or a floppy disk), an optical disk (for example, a CD-ROM, a DVD-ROM, or a Blu-ray (registered trademark) disk), a magneto-optical disk (for example, an MO), or a flash memory (for example, an SD card, a USB memory, or an SSD). In this case, the physiological information processing program stored in the storage medium may be incorporated into the storage device 3. Further, the program incorporated in the storage device 3 may be loaded onto the RAM, and then the processor may execute the program loaded onto the PAM. In this way, a physiological information processing method according to the present embodiment is executed by the processing device 1.

The physiological information processing program may also be downloaded from a computer on a communication network via the network interface 4. In this case, same or similarly, the downloaded program may be incorporated into the storage device 3.

Although the embodiment of the presently disclosed subject matter has been described above, the technical scope of the presently disclosed subject matter should not be construed as being limited by the description of the embodiment. It is to be understood by those skilled in the art that the present embodiment is an example and various modifications can be made within the scope of the invention described in the claims. The technical scope of the presently disclosed subject matter should be determined based on the scope of the invention described in the claims and the scope of equivalents thereof.

In the present embodiment, the trend graphs of the cardiac output CO, the cardiac output index CI, and the stroke volume SV that serve as the parameters related to the amount of blood delivered from the heart of the subject P have been described. However, the processing device 1 may display a trend graph indicating a change over time in a parameter related to stiffness of a blood vessel of the subject P in the graph display region 100. For example, the processing device 1 may display, in the graph display region 100, a trend graph indicating a change over time in systemic vascular resistance SVR or systemic vascular resistance index SVRI that is an example of the parameter related to the stiffness of the blood vessel of the subject P.

In this case, the control unit 2 acquires the blood pressure data indicating the change over time in the arterial pressure and the blood pressure data indicating the change over time in the venous pressure from the blood pressure sensor 16. Thereafter, the control unit 2 specifies the systolic blood pressure and the diastolic blood pressure based on the blood pressure data indicating the change over time in the arterial pressure, and then acquires mean blood pressure IBPₘₑₐₙ based on the systolic blood pressure and the diastolic blood pressure. In addition, the control unit 2 specifies central venous pressure CVP based on the blood pressure data indicating the change over time in the venous pressure. Next, the control unit 2 calculates the systemic vascular resistance SVR based on the mean blood pressure IBPₘₑₐₙ, the central venous pressure CVP, the calculated cardiac output CO, and the following relational expression (6).

SVR=(IBPₘₑₐₙ·CVP)×80/CO (6)

The control unit 2 calculates the systemic vascular resistance index SVRI based on the calculated SVR and the following relational expression (7). As described above, BSA (m²) corresponds to the body surface area of the subject P determined based on the height and weight of the subject P.

SVRI=SVR/BSA (7)

Thereafter, the control unit 2 updates the trend graph of the SVR or the trend graph of the SVRI. The trend graph indicating the change over time in the SVR or the SVRI and the trend graph indicating the change over time in the SVV may be simultaneously displayed in the graph display region 100. Further, the reference guideline 30 indicating a reference value of the SVR or the SVRI and the reference guide region 40 indicating the predetermined range from the reference value of the SVR or the SVRI may be displayed in the graph display region 100.

In the present embodiment, the parameter such as the cardiac output CO is calculated based on the physiological information data (electrocardiogram data or the like) acquired from the physiological information sensor. On the other hand, the parameter such as the cardiac output CO may be directly acquired by a cardiac output sensor such as a Swan-Ganz catheter. As described above, the parameter such as the cardiac output CO may be calculated based on the physiological information data, or may be directly acquired from the physiological information sensor such as the cardiac output sensor.

What is claimed is:

1. A physiological information processing method comprising:
acquiring a parameter related to an amount of blood delivered from a heart of subject or stiffness of a blood vessel of the subject;
displaying a trend graph indicating a change over time in the parameter in a graph display region;
determining a reference value of the parameter in accordance with an input operation of a user; and
displaying a reference guideline indicating the reference value of the parameter in the graph display region.

2. The physiological information processing method according to claim 1, further comprising:
acquiring at least one piece of physiological information data of the subject from a physiological information sensor, wherein
the acquiring the parameter includes: calculating the parameter based on the at least one piece of the physiological information data.

3. The physiological information processing method according to claim 1, further comprising:
determining a predetermined range from the reference value in accordance with the input operation of the user; and
displaying a reference guide region indicating the predetermined range from the reference value in the graph display region.

4. The physiological information processing method according to claim 1, wherein the parameter includes a stroke volume variation of the subject,
the trend graph includes:
a first trend graph indicating a change over time in the parameter other than the stroke volume variation; and
a second trend graph indicating a change over time in the stroke volume variation, and
the displaying the trend graph includes: simultaneously displaying the first trend graph and the second trend graph in the graph display region.

5. The physiological information processing method according to claim 4, wherein the parameter further includes:
a cardiac output of the subject;
a stroke volume of the subject; and
a cardiac output index of the subject, and
the first trend graph is any one of
a trend graph indicating a change over time in the cardiac output,
a trend graph indicating a change over time in the stroke volume, and
a trend graph indicating a change over time in the cardiac output index.

6. The physiological information processing method according to claim 5, wherein the reference value of the parameter is a reference value of the cardiac output, the stroke volume, or the cardiac output index.

7. The physiological information processing method according to claim 1, wherein the determining the reference value is performed before a fluid is administered to the subject, and
the reference value is a value of the parameter acquired before the fluid is administered to the subject.

8. A computer-readable storage medium storing a program configured to cause a computer to execute the physiological information processing method according to claim 1.

9. A physiological information processing device comprising:
one or more processors; and
one or more memories configured to store a computer readable command, wherein
when the computer readable command is executed by the one or more processors, the physiological information processing device is configured to:
acquire a parameter related to an amount of blood delivered from a heart of a subject or stiffness of a blood vessel of the subject,
display a trend graph indicating a change over time in the parameter in a graph display, region,
determine a reference value of the parameter in accordance with an input operation of a user, and
display a reference guideline indicating the reference value of the parameter in the graph display region.

10. The physiological information processing device according to claim 9, wherein the physiological information processing device is configured to:
acquire at least one piece of physiological information data of the subject from a physiological information sensor, and
calculate the parameter based on the at least one piece of the physiological information data.

11. The physiological information processing device according to claim 9, wherein the physiological information processing device is configured to:
determine a predetermined range from the reference value in accordance with the input operation of the user, and
display a reference guide region indicating the predetermined range from the reference value in the graph display region.

12. The physiological information processing device according to claim 9, wherein the parameter includes a stroke volume variation of the subject,
the trend graph includes:
a first trend graph indicating a change over time in the parameter other than the stroke volume variation; and
a second trend graph indicating a change over time in the stroke volume variation, and
the physiological information processing device simultaneously displays the first trend graph and the second trend graph in the graph display region.

13. The physiological information processing device according to claim 12, herein the parameter further includes:
a cardiac output of the subject;
a stroke volume of the subject; and
a cardiac output index of the subject, and
the first trend graph is any one of
a trend graph indicating a change over time in the cardiac output,
a trend graph indicating a change over time in the stroke volume, and
a trend graph indicating a change over time in the cardiac output index.

14. The physiological information processing device according to claim 13, wherein the reference value of the parameter is a reference value of the cardiac output, the stroke volume, or the cardiac output index.

15. The physiological information processing device according to claim 9, wherein the physiological information processing device determines the reference value before a fluid is administered to the subject, and
the reference value is a value of the parameter acquired before the fluid is administered to the subject.
